(19) United States
(12) Patent Application Publication
(10) Pub. No.: US 2006/0099261 A1
(43) Pub. Date: May 11, 2006

(54) Methods and formulations for making controlled release oral dosage form

(75) Inventors: San-Laung Chow, San Jose, CA (US); David Wong, Milpitas, CA (US); Damian Garcia, Sunnyvale, CA (US)

Correspondence Address: PATTERSON & SHERIDAN, L.L.P., 3040 POST OAK BOULEVARD, SUITE 1500, HOUSTON, TX, 77056, US

(73) Assignee: BioKey, Inc.

(21) Appl. No.: 11/295,851

(22) Filed: Dec. 6, 2005

Related U.S. Application Data
(63) Continuation-in-part of application No. 11/265,918, filed on Nov. 3, 2005.
(60) Provisional application No. 60/626,317, filed on Nov. 8, 2004.

(51) Int. Cl. A61K 9/24

(52) U.S. Cl. 424/472

(57) ABSTRACT

Embodiments of the invention generally provide pharmaceutical drug compositions, methods of preparing oral drug compositions, such as extended release dosage compositions, and methods for treating antidepressant or smoking cessation. In one aspect, the invention provides a pharmaceutical formulation comprising a core, including bupropion and its salt derivatives, and a coating. The coating may include one or more surfactants and aqueous dispersions of one or more insoluble pharmaceutical acceptable polymers. The core may also include aqueous dispersions of one or more insoluble pharmaceutical acceptable polymers. In another aspect, the invention provides methods for preparing and administering a pharmaceutical composition in oral dosage form, such as a tablet.

### CROSS-REFERENCE TO RELATED APPLICATIONS

This application is a continuation-in-part of co-pending U.S. patent application Ser. No. 11/265,918, filed Nov. 3, 2005, which claims benefit of U.S. provisional patent application Ser. No. 60/626,317, entitled, “METHODS AND FORMULATIONS FOR MAKING PHARMACEUTICAL COMPOSITIONS CONTAINING BUPROPION”, filed Nov. 8, 2004. Each of the aforementioned related patent application is herein incorporated by reference.

### BACKGROUND OF THE INVENTION

The invention generally relates to pharmaceutical compositions, such as drug formulations present in a solid form for oral administration. More particularly, the invention relates to long-lasting sustained dosage compositions, and carriers and active ingredients in the compositions thereof, such as controlled release and extended release drug compositions for oral controlled release dosage formulations containing a drug and a carrier material.

Drug delivery at a predetermined rate such that drug concentrations can be maintained at desired therapeutically effective levels over an extended period, has received a great deal of attention. Many known solid drug formulations are required to be taken orally three or four times a day. There is a need for oral formulations to be taken less often, such as once per day. In addition, there are other problems with undesired drug delivery rate. For example, various side effects are observed for immediate release drug formulations due to high drug concentrations released in the plasma or blood stream right after the intake of the drug.

Bupropion is commonly used as an antidepressant drug. It is generally formulated from its salt derivatives, such as its hydrochloride salts. A commercial example is Wellbutrin®. However, it has been shown that immediate release formulations of bupropion hydrochloride can induce some severe side effects, such as seizures, high blood pressure, and severe allergic reactions. There is a need to prepare a new sustained release dosage form to reduce side effect problems.

Various approaches exist for preparing sustained or controlled release pharmaceutical formulations, such as various extended release formulations in tablet or capsule form. For example, one method of forming delayed or sustained release formulations includes coating the tablet with a release-retarding coating, or coating individual granules with such a coating, and compressing these coated granules into a tablet. Exemplary techniques involving sustained release solid preparations for bupropion hydrochloride in a matrix are described in U.S. Pat. Nos. 5,358,970 and 5,427,798. However, bupropion hydrochloride is unstable and a stabilizer, such as cysteine hydrochloride, glycine hydrochloride, malic acid, citric acid, cystine dihydrochloride, etc., as described in the above two patents is required to stabilize the drug and thus making the technique not very well suited for manufacturing.

Another example involves controlled release tablet formulations for bupropion hydrochloride by using a core containing bupropion hydrochloride and a coating of a mixture having a film-forming polymer, a pore-forming agent, and other excipients, as described in U.S. Pat. No. 4,687,660 and EP-A-0171457. However, the pore-forming agent, such as sodium carbonate, renders the coating of the core non-uniform and the release rate of the tablet not stable. Other examples of controlled release tablets, as described in U.S. Pat. Nos. 6,033,686; 6,096,341 and 6,143,327, require a water-insoluble/water-permeable film-forming polymer in a first coating solution to coat a drug-containing core and prepare a film coated tablet. In these systems, the water-insoluble film-forming polymer in the first coating solution is very thermodynamically unstable and tends to aggregate rapidly, resulting in clotting problems during spray-coating the drug-containing core; and plasticizers, such as polyethylene glycols (PEG), are often required to soften the film coated tablet. A second coating or an immediate release coating is also required to coat the film coated tablet to form a delayed release tablet.

Therefore, there is a need for an improved controlled release formulation and method for preparing such a controlled release formulation.

### SUMMARY OF THE INVENTION

The invention generally provides a pharmaceutical composition having one or more pore-forming materials without the aid of any plasticizers. In one aspect, the pharmaceutical composition includes one or more surfactants in a coating mixture to create channels or pores in a coating layer for the release of a drug from a drug-containing core. In another aspect, the pharmaceutical composition includes one or more aqueous polymeric colloidal dispersions adapted to coat a drug-containing core. Optionally, one or more aqueous polymeric colloidal dispersions can also be adapted to granulate a drug and included into the core.

In one embodiment, the pharmaceutical composition includes a therapeutically active agent, such as bupropion and its salts and derivatives thereof, prepared into a core. The pharmaceutical composition may further include a coating outside the core. The coating may include a surfactant and an aqueous dispersion of one or more insoluble pharmaceutical acceptable polymers, which may be one or more pH-independent and pH-dependent polymers.

In another embodiment, a pharmaceutical composition which includes a bupropion salt containing core and a coating is provided. The coating may include one or more surfactants and aqueous dispersions of one or more insoluble pharmaceutical acceptable polymers. The one or more insoluble pharmaceutical acceptable polymers may include one or more pH-independent polymers, such as swellable permeable neutral ester copolymer dispersions. The one or more insoluble pharmaceutical acceptable polymers may also include one or more pH-dependent polymers, such as ionic polymer dispersions.

In another embodiment, an extended release pharmaceutical composition is provided and includes a core of a pharmaceutical mixture containing bupropion salt and a first aqueous dispersion of one or more insoluble pharmaceutical acceptable polymers, and a coating layer. The coating layer for the core of the pharmaceutical mixture may include one or more surfactants and a second aqueous dispersion of one or more insoluble pharmaceutical acceptable polymers.

In still another embodiment, the invention provides a pharmaceutical composition including: a core and a coating. The core may include a bupropion salt and a first swellable permeable insoluble polymer dispersion. The coating may include one or more surfactants and a second swellable permeable insoluble polymer dispersion. The pharmaceutical composition may further comprise an insoluble ionic polymer dispersion.

In yet another embodiment, the invention further provides a method of preparing a pharmaceutical composition. The method includes forming a core of a pharmaceutical mixture comprising bupropion salt, and coating the core with a coating mixture. The coating mixture may include one or more surfactants and aqueous dispersions of one or more insoluble pharmaceutical acceptable polymers.

In yet another embodiment, a method of administering a pharmaceutical composition containing bupropion salt is provided. The method includes administering to a mammal an effective amount of the pharmaceutical composition comprising a bupropion salt containing core and a coating mixture. The coating mixture includes one or more surfactants and aqueous dispersions of one or more insoluble pharmaceutical acceptable polymers.

### BRIEF DESCRIPTION OF THE DRAWINGS

So that the manner in which the above recited features, advantages and objects of the present invention are attained and can be understood in detail, a more particular description of the invention, briefly summarized above, may be had by reference to the embodiments thereof which are illustrated in the appended drawings. It is to be noted, however, that the appended drawings illustrate only typical embodiments of this invention and are therefore not to be considered limiting of its scope, for the invention may admit to other equally effective embodiments.

FIG. 1 is a release profile of an exemplary pharmaceutical composition according to one embodiment of the invention.

FIG. 2 is a release profile of an exemplary pharmaceutical composition according to another embodiment of the invention.

FIG. 3 is a release profile of an exemplary pharmaceutical composition according to still another embodiment of the invention.

FIG. 4 is a release profile of an exemplary pharmaceutical composition according to still another embodiment of the invention.

### DETAILED DESCRIPTION OF THE INVENTION

The pharmaceutical composition of the invention includes a therapeutically active agent, a surfactant, and a pharmaceutically acceptable pH-independent polymer without any stabilizer or any plasticizer. The pharmaceutical composition is generally prepared into an oral dosage form or a solid dosage form, such as a tablet, a capsule, a sachet, etc., and any therapeutically acceptable form. The invention provides a method of stabilizing a therapeutically active agent using a coating layer which contains a surfactant or oil to seal away any factors affecting the stability of the therapeutically active agent in an coating layer and create channels and pores during the release of the therapeutically active agent. However, it is very difficult to incorporate a liquid material, such as surfactants or oils, into a solid dosage form, especially a tablet dosage form. Most pharmaceutical polymers can not absorb liquid materials since the liquids materials tend to be squeezed out of the solid dosage form, resulting in solid dosage forms with reduced compactibility and compressibility.

One embodiment of the invention provides using an aqueous dispersion of one or more insoluble pharmaceutical acceptable polymers or polymeric colloidal dispersions adapted to absorb a liquid material, such as surfactants or oils, into a solid dosage form without the use of any plasticizers. Another embodiment of the invention provides using an aqueous dispersion of one or more insoluble pharmaceutical acceptable polymers to granulate a therapeutically active agent in a pharmaceutical composition.

Various insoluble pharmaceutical acceptable polymers are available in aqueous dispersion and include, but are not limited to, polyacrylates, various copolymers of acrylate and methacrylates, copolymers of ionic methacrylates and neutral methacrylates, cellulose derivatives (e.g., ethylcellulose), polyvinyl acetate (e.g., Kollicoat SR30D from BASF Corporation, Mount Olive, N.J.), neutral copolymers based on ethyl acrylate and methylmethacrylate, ionic polymers based on methacrylic acid and methacrylate, methacylic acid copolymers, ionic polymers based on methacrylates with various side chains or functional groups (e.g., ammonium group, carboxylic acid groups, or dimethylaminoethyl groups), poly(meth)acrylate polymers with acidic functional groups, poly(meth)acrylate polymers with alkaline functional groups, hydroxypropyl methylcellulose phthalate, hydroxypropyl methylcellulose acetate, hydroxypropyl methylcellulose succinate, carboxymethylethylcellulose, cellulose acetophthalate, among others.

The aqueous dispersion of the one or more insoluble pharmaceutical acceptable polymers may include one or more pH-independent polymers, such as swellable permeable neutral ester copolymer dispersions. The aqueous dispersion of the one or more insoluble pharmaceutical acceptable polymers may include one or more pH-dependent polymers, such as ionic polymer dispersions. One example of an insoluble pharmaceutical acceptable polymer includes copolymers of acrylate and methacrylates with quaternary ammonium groups, such as Eudragit® RS, RS 30 D, RL, RL 30 D, and the like (Röhm America, LLC). Another example includes neutral ester copolymers of acrylates, such as Eudragit® NE 30 D, NE 40 D, and the like. Still, another example includes ionic polymers of methacrylates, such as Eudragit® L, S, FS, and E, and the like (Röhm America, LLC).

The aqueous dispersion of the one or more insoluble pharmaceutical acceptable polymers may be used to absorb one or more surfactants or oils in the pharmaceutical composition. The surfactant, oil, and detergent of the invention include any of the pharmaceutically acceptable or medicinally acceptable ionic, anionic, cationic, or non-ionic surfactant or detergent. For example, one or more of sodium lauryl sulfate, polysorbates, Tween 80 (available form Fisher Scientific International), Tween 20, Tween 60, Tween 100, and others can be used to a concentration of no more than 50% of the total weight, such as from about 0.01% to about 10% of the total weight of the pharmaceutical composition. Not wishing to be bound by the theory, it is contemplated that the use of one or more surfactants creates channels or pores in a coating layer such that water and other media can get into a drug-containing core during the release of the drug. It is thought that the use of polymeric colloidal dispersions or aqueous dispersions of the one or more insoluble pharmaceutical acceptable polymers together with the one or more channel-forming surfactants eliminates the need to use a plasticizer, or any plasticizers which are also used as pore-forming materials, e.g., polyethylene glycol, etc.

The aqueous dispersion of the one or more insoluble pharmaceutical acceptable polymers may also be used to granulate the therapeutically active agent in the pharmaceutical composition. The pharmaceutical composition of the invention generally includes the therapeutically active agent utilized at therapeutic dose levels, which may vary from about 5% to about 95% by weight of the pharmaceutical composition, preferably, from about 30% to about 90% by weight of the pharmaceutical composition. One example of the therapeutically active agent includes bupropion and its salts and derivatives thereof, such as bupropion hydrochloride at a concentration of about 45% to about 85% by weight. For example, about 10 mg to about 500 mg of bupropion hydrochloride can be prepared into the pharmaceutical composition of the invention without any stabilizer or any plasticizer and still maintaining its stability for long term storage, even under elevated storage conditions.

Other therapeutically active agents can also be used herein, including, but not limited to, various water-soluble drugs, water-insoluble drugs, valproic acid, benzonatate, simethicone, methylclothiazide, prednisolone, prednisone, ibuprofen, naproxen, aspirin, acetominophen, dihydroergotamine mesylate, famotidine, omeprasole, chloropheniramine, ranitidine, diclofenac sodium, cimetidine, guaifenesin, glypizide, estradiol, acyclovir, ketoprofen, desmopressin, oxybutynin hydrochloride, propranolol hydrochloride, niacin, cetirizin hydrochloride, cerivastin sodium, metoprolol fumarate, nifedipine, nisoldipine, nicardipine, nilvadipine, felodipine, bendroflumethazide, acetazolamide, methazolamide, chlorpropamide, methotrexate, allopurinol, hydrocortisone, triamcinolone, prednisone, prednisolone, norgestrel, norethindone, progesterone, norgesterone, atenolol, timolol, cimetidine, clonidine, and alendronate sodium. Other suitable therapeutically active agents may be an active ingredient compound having a therapeutic effect and including antibiotics, anti-infection agents, anti-hypertensives, calcium channel blockers, β-blockers, analgesics, anti-neoplastic agents, anti-microbials, anti-malarials, non-steroidal anti-inflammatory drugs (NSAID), diuretics, anti-arrythmia agents and the like. The therapeutically active agent can be prepared into powder, granules, particles, beads, pellets, and other pharmaceutical acceptable sizes. The therapeutically active agent can further be micronized and preferably have a particle size of less than 20 microns.

In one embodiment, the invention provides a pharmaceutical composition comprising a therapeutically active agent prepared into a core which can be coated with a pharmaceutical acceptable coating mixture. The core may further include an aqueous dispersion of one or more insoluble pharmaceutical acceptable polymers of the invention adapted to granulate a drug. In addition, the pharmaceutical acceptable coating mixture may include one or more surfactants and an aqueous dispersion of one or more insoluble pharmaceutical acceptable polymers adapted to help absorbing the one or more surfactants or detergents and coat the core. The aqueous dispersion of the one or more insoluble pharmaceutical acceptable polymers may each be used to a concentration from about 0.001% to about 50% by weight of the total weight of the pharmaceutical composition.

The one or more insoluble pharmaceutical acceptable polymers in the core may be the same or different polymers as the one or more insoluble pharmaceutical acceptable polymers in the pharmaceutical acceptable coating mixture. The one or more insoluble pharmaceutical acceptable polymers may include pH-dependent polymers and/or pH-independent polymers. For example, an aqueous dispersion of a swellable permeable insoluble pharmaceutical acceptable polymer, which may be a pH-dependent polymer or a pH-independent polymer, can be incorporated into the core of the pharmaceutical composition of the invention. Preferably, aqueous dispersions of one or more pH-independent polymers are included in the core to mix with the therapeutically active agent of the invention. Exemplary aqueous dispersions of swellable permeable insoluble pH-independent polymers in the core include Eudragit® NE 30 D, NE 40 D, and the like.

As one example, aqueous dispersions of one or more swellable permeable neutral ester copolymers are included in the core. The same swellable permeable insoluble pharmaceutical acceptable polymer in the core can also be incorporated into the pharmaceutical acceptable coating mixture. In this embodiment, the pharmaceutical composition of the invention may further include additional aqueous dispersions of insoluble pharmaceutical acceptable polymers, which may be a pH-dependent polymer or a pH-independent polymer, incorporated into the pharmaceutical acceptable coating mixture. Preferably, one or more swellable permeable neutral ester copolymer dispersions and ionic polymer dispersions are included in the pharmaceutical acceptable coating mixture. The presence of the ionic polymer dispersions in the pharmaceutical acceptable coating mixture may aid the dissolution of the therapeutically active agent under various in vitro and in vivo conditions which may require different pH or ionic interactions.

As another example, aqueous dispersions of one or more insoluble pharmaceutical acceptable enteric polymers are included in the pharmaceutical acceptable coating mixture. Examples of enteric polymers include methacylic acid copolymers (e.g., Eudragit® S and Eudragit® L, available from Röhm America, LLC), hydroxypropyl methylcellulose phthalate, hydroxypropyl methylcellulose acetate, hydroxypropyl methylcellulose succinate, carboxymethylethylcellulose, cellulose acetophthalate. The one or more insoluble pharmaceutical acceptable enteric polymers may be pH-dependent ionic polymers which are only soluble under certain pH levels. In this embodiment, the pharmaceutical composition of the invention may further include additional aqueous dispersions of insoluble pharmaceutical acceptable polymers, which may be a pH-dependent polymer or a pH-independent polymer, incorporated into the core and/or the pharmaceutical acceptable coating mixture.

In another embodiment, the invention provides extended release formulations for the therapeutically active agent. For example, the pharmaceutical composition of the invention includes a controlled release, sustained release, or timed release dosage formulation for the therapeutically active agent. The extended release formulation as described herein can provide continuous and non-pulsating therapeutic levels of the therapeutically active agent to a mammal in need of such treatment over a period of time, such as a four-hour period or longer, a six-hour period or longer, e.g., a twelve-hour to twenty-four hour period. Such an extended release, controlled release, sustained release, or timed release dosage formulation employs a core of a pharmaceutical mixture and a coating layer of a pharmaceutical acceptable coating mixture.

The core of the pharmaceutical mixture contains the therapeutically active agent (e.g., bupropion hydrochloride) and optionally, includes aqueous dispersions of one or more insoluble pharmaceutical acceptable polymers of the invention at a concentration of from about 0.01% to about 80% of the total weight of the pharmaceutical composition, such as from about 0.01% to about 50% by weight. The core containing the therapeutically active agent generally includes a therapeutically active agent and aqueous dispersions of one or more insoluble pharmaceutical acceptable polymers, which may help swelling or gelling of the therapeutically active agent and carrying the therapeutically active agent to be released in a controlled or time-delayed manner. Exemplary aqueous dispersions of the one or more insoluble pharmaceutical acceptable polymers in the core include swellable permeable neutral ester copolymer dispersions, such as Eudragit® NE 30 D, NE 40 D, and the like, used to a concentration of from about 0.01% to about 50% by weight of the total weight of the pharmaceutical concentration.

The core of the pharmaceutical mixture may optionally include water-soluble polymers. Examples of water-soluble polymers include polyvinylpyrrolidone, hydroxypropyl cellulose (HPC; Klucel), hydroxypropyl methylcellulose (HPMC; Methocel), nitrocellulose, hydroxypropyl ethylcellulose, hydroxypropyl butylcellulose, hydroxypropyl pentylcellulose, methyl cellulose, ethylcellulose (Ethocel), hydroxyethyl cellulose, various alkyl celluloses and hydroxyalkyl celluloses, various cellulose ethers, cellulose acetate, carboxymethyl cellulose, sodium carboxymethyl cellulose, calcium carboxymethyl cellulose, vinyl acetate/crotonic acid copolymers, poly-hydroxyalkyl methacrylate, hydroxymethyl methacrylate, methacrylic acid copolymers, polymethacrylic acid, polymethylmethacrylate, maleic anhydride/methyl vinyl ether copolymers, poly vinyl alcohol, sodium and calcium polyacrylic acid, polyacrylic acid, acidic carboxy polymers, carboxypolymethylene, carboxyvinyl polymers, polyoxyethylene polyoxypropylene copolymer, polymethylvinylether co-maleic anhydride, carboxymethylamide, potassium methacrylate divinylbenzene co-polymer, polyoxyethyleneglycols, polyethylene oxide, and derivatives, salts, and combinations thereof.

The core of the pharmaceutical mixture can be prepared in a form of granules, particles, beads, spherical beads, pellets, coated beads, coated pellets, coated particles, and other pharmaceutically acceptable shapes and sizes. This can be done by various granulation methods and other methods, such as wet and dry granulations. Wet granulation is prepared by mixing required components with various conventional well-known solvents to form granules. Alternatively, dry granulation techniques may be used to prepare the pharmaceutical composition. The mixture of the core of the pharmaceutical composition can then be incorporated into solid dosage forms, such as tablets and others, and an optional external coating is applied. For making compressed tablets, a conventional tabletting machine may be used to compress a granulated mixture of the components of the present invention into a tablet.

Embodiments of the invention provide a coating layer outside of the core containing the therapeutically active agent. The coating layer of the pharmaceutical acceptable coating mixture includes one or more surfactants and aqueous dispersions of one or more insoluble pharmaceutical acceptable polymers of the invention. Ionic surfactant, anionic surfactants, non-ionic surfactants, emulsifiers, dispersing agents, defoamers, and combinations thereof can be used to advantage. Any of the pharmaceutically acceptable or medicinally acceptable surfactants, emulsifiers, dispersing agents, dispersants, and defoamers can be used herein at a concentration of no more than 50% of the total weight of the pharmaceutical concentration, such as from about 0.01% to about 10% of the total weight. For example, a surfactant can be used in the coating layer of the pharmaceutical composition to an amount varying from about 0.001% to about 50% by weight of the coating, such as from about 0.1% to about 30% by weight of the coating. Not wishing to be bound by any theory, it is believed that the surfactant in the pharmaceutical composition enhances the channeling process of the one or more insoluble pharmaceutical acceptable polymers to achieve a desired drug release profile. One exemplary surfactant includes sodium lauryl sulfate, used to a concentration of no more than 50% of the total weight of the pharmaceutical concentration or from about 0.01% to about 30% by weight of the coating layer of the pharmaceutical acceptable coating mixture. Optional, a second surfactant, such as Tween 80 Tween 20, Tween 60, Tween 100, and others can also be included in the pharmaceutical acceptable coating mixture.

Exemplary aqueous dispersions of the one or more insoluble pharmaceutical acceptable polymers in the coating layer of the invention include swellable permeable neutral ester copolymer dispersions, such as copolymers of acrylate and methacrylates neutral esters, Eudragit® NE 30 D, NE 40 D, and the like, used to a concentration of from about 0.01% to about 50% by weight of the total weight of the pharmaceutical concentration, such as from about 0.01% to about 10% by weight of the pharmaceutical composition. In addition, the one or more insoluble pharmaceutical acceptable polymers in the coating layer can include ph-independent or pH-dependent polymers, used at a concentration from about 0.01% to about 99% by weight of the coating layer of the pharmaceutical acceptable coating mixture, e.g., from about 5% to about 99% by weight of the coating layer. Other pH-independent polymers, such as copolymers of acrylate and/or methacrylate can also be used to advantage; for example, Eudragit® RL, Eudragit® RS and the like. Other representative examples of water-insoluble polymers useful in the invention include polyacrylates, cellulose derivatives (e.g., ethylcellulose), polyvinyl acetate (e.g., Kollicoat SR30D from BASF Corporation, Mount Olive, N.J.), neutral copolymers based on ethyl acrylate and methylmethacrylate, copolymers of acrylate and methacrylates, among others.

In addition, additional aqueous dispersions of insoluble pharmaceutical acceptable polymers, such as pH-dependent enteric polymers, can also be included in the pharmaceutical acceptable coating mixture. Exemplary aqueous dispersions of the pH-dependent enteric polymers in the coating layer of the invention include Eudragit® L 30 D-55, FS 30D, and the like, used to a concentration of from about 0.01% to about 50% by weight of the total weight of the pharmaceutical concentration, such as from about 0.01% to about 10% by weight of the pharmaceutical composition. The one or more insoluble pH-dependent polymers in the coating layer may be used at a concentration from about 0.01% to about 99% by weight of the coating layer of the pharmaceutical acceptable coating mixture, e.g., from about 5% to about 99% by weight of the coating layer. Other pH-dependent ionic polymers, such as Eudragit® L 12,5 or S 12,5, and the like, can also be used to advantage. Examples of enteric polymers include methacylic acid copolymers (e.g., Eudragit® S and Eudragit® L, available from Röhm America, LLC), hydroxypropyl methylcellulose phthalate, hydroxypropyl methylcellulose acetate, hydroxypropyl methylcellulose succinate, carboxymethylethylcellulose, cellulose acetophthalate. Generally, enteric polymers rapidly disintegrate or dissolve at pH 5 or above. Other suitable enteric polymers may include cellulose acetate phthalate, polyvinyl acetate phthalate, acrylic resins, shellac, cellulose acetate butyrate, hydroxypropyl methylcellulose phthalate, cellulose acetyl phthalate, cellulose triacetyl phthalate, sodium cellulose acetate phthalate, cellulose ester phthalate, cellulose ether phthalate, methylcellulose phthalate, cellulose ester-ether phthalate, hydroxy propyl cellulose phthalate, alkali salts of cellulose acetate phthalate, alkaline earth salts of cellulose acetate phthalate, calcium salt of cellulose acetate phthalate, ammonium salt of hydroxypropyl methylcellulose phthalate, cellulose acetate hexahydrophthalate, hydroxypropyl methylcellulose hexahydrophthalate, polyvinyl acetate phthalate, and combinations thereof. The enteric materials are discussed in Remington's Pharmaceutical Sciences, 17th Ed., page 1637 (1985.

The core of the pharmaceutical mixture and the coating layer of the pharmaceutical acceptable coating mixture may further include pharmaceutically acceptable excipients, fillers, diluents, binders, and blending agents, such as hydrous or anhydrous form of lactose, starches, glucose, sucrose, mannitol, sorbitol, silicic acid, microcrystalline celluloses, sodium carboxymethylcelluloses, sodium starch glycolate, and derivatives and mixtures thereof. For example, avicel can be added into the core to a concentration of from about 0.01% to about 50% by weight of the pharmaceutical composition, such as from about 0.05% to about 40% by weight. The core and coating layer of the invention may further include glidants, lubricants disintegrants, flavours, colourants, antiadherents, blenders, anti-sticking agents, wetting agents, dyes, pigments, nonstick agents, dispersants, coating materials, and mixtures thereof, to be combined into the pharmaceutical composition. Examples of lubricants include, but are not limited to, talc, calcium stearate, magnesium stearate, glycerol monostearate, polyethylene glycols, inert silicon glass materials, colloidal silicon dioxide, and higher fatty acids and their alkali-metal and alkaline-earth-metal salts. In addition, various excipients such as diluents, lubricants, dyes, etc., which are disclosed in Remington's Pharmaceutical Sciences, 1995 Edition, may be used to optimize the pharmaceutical composition of the invention. The amounts of the lubricants, anti-sticking agents, and other excipients generally vary from about 0.005% to about 50% by weight of the pharmaceutical composition, such as from about 0.005% to about 30%. It is found that the release rate of the therapeutically active agent can be controlled not only by incorporating suitable insoluble pharmaceutically acceptable polymers and surfactants therein, but also by the thickness of the coating layer having excipients applied.

Examples that can be blended into the core and coating layer of the pharmaceutical composition include glycerol monosteartae, talc, corn starch, magnesium stearate, cab-O-sil, and avicel, to a final concentration of from about 1.0% to about 20% by weight of the pharmaceutical composition. For example, glycerol monosteartae can be added into the core and/or the coating layer to a concentration of from about 0.01% to about 50% by weight of the pharmaceutical composition, such as from about 0.05% to about 40% by weight of the pharmaceutical composition.

The pharmaceutical compositions of the invention may have an additional second coating layer containing a pharmaceutically acceptable coating mixture. The pharmaceutically acceptable coating mixture in the second coating layer includes, but is not limited to, an enteric polymer, a salt, a rapid-disintegrating coating material, a colorant, a water-soluble polymer, a water-insoluble polymer, a dye, a pigment, other disintegrants, and combinations thereof. One common example of rapid-disintegrating coating material is OPADRY, available from Colorcon, Inc. Any commonly used pharmaceutically acceptable salts can be used in the coating layer, such as the first coating layer or the second coating layer. For example, sodium chloride, magnesium choride, among others. Generally, the amount of the second coating layer surrounding the coated tablet is from about 0.001% to about 5% of the total weight of the pharmaceutical composition, such as from about 0.01% to about 2% based on the total weight of the pharmaceutical composition.

In one embodiment, the invention provides prolonged release formulations of bupropion hydrochloride prepared by the pharmaceutical composition of the invention, such as controlled release or extended release formulations. In an alternative embodiment, the invention provides a method of preparing a pharmaceutical composition, such as an extended release formulation, by preparing a core and coating the core with the coating layer. The coated core is then incorporated into a dosage form, such as by compressing the pharmaceutical composition into an oral dosage form, a solid dosage form, and a tablet dosage form.

For example, a method of preparing the pharmaceutical composition may include forming a core of a pharmaceutical mixture and coating the core with a coating mixture. The pharmaceutical mixture may include, for example, bupropion, such as bupropion salt, e.g., bupropion hydrochloride and the core of the pharmaceutical mixture may optionally include aqueous dispersions of one or more insoluble pharmaceutical acceptable polymers. The coating mixture may include one or more surfactants and aqueous dispersions of one or more insoluble pharmaceutical acceptable polymers of the invention.

In operation, the core may be formed by mixing the pharmaceutical mixture through granulation, such as wet granulation or dry granulation, compressing the pharmaceutical mixture into a dosage form, including an oral dosage form, a solid dosage form, and a tablet dosage form, etc. For example, the pharmaceutical mixture may be mixed through wet granulation and compressed into a tablet core. In addition, a coating mixture is formed by mixing one or more surfactants with aqueous dispersion of one or more insoluble pharmaceutical acceptable polymers of the invention, and the tablet core is coated with the coating mixture. Coating a core with the coating mixture of the invention may employ any suitable pharmaceutical coating techniques, such as press coating, molding, spraying, dipping and/or air-suspension or air tumbling procedures. One example of coating a core with a coating mixture includes pan coating, where the coating mixture is applied by spraying onto the tablet core accompanied by tumbling in a rotating pan.

Accordingly, an extended release pharmaceutical composition of the invention may include a core of a pharmaceutical mixture comprising from about 10 mg to about 500 mg of bupropion hydrochloride and a coating layer comprising one or more surfactants and one or more aqueous water-insoluble pharmaceutical acceptable polymeric colloidal dispersions. The core may further include a pharmaceutical acceptable polymer, for example, a water-soluble polymer, a water-insoluble polymer, a pH-independent polymer, a pH-dependent polymer, and any of the combinations thereof. Preferably, an aqueous water-insoluble pharmaceutical acceptable polymeric dispersion can be incorporated to the core such that the final solid dosage form of the bupropion hydrochloride is not too brittle. Examples of suitable aqueous water-insoluble pharmaceutical acceptable polymeric dispersions which can be incorporated into the core include various polymeric colloidal dispersions, such as pH-independent polymeric colloidal dispersions, pH-dependent polymeric colloidal dispersions, swellable permeable neutral ester copolymer colloidal dispersions, ionic polymeric colloidal dispersions, and any of the combinations thereof.

The coating layer of the extended release pharmaceutical composition of the invention may include one or more aqueous water-insoluble pharmaceutical acceptable polymeric colloidal dispersions, such as various water-insoluble polymeric colloidal dispersions, e.g., pH-independent water-insoluble polymeric colloidal dispersions, pH-dependent water-insoluble polymeric colloidal dispersions, water-insoluble swellable permeable neutral ester copolymer colloidal dispersions, water-insoluble ionic polymeric colloidal dispersions, and any of the combinations thereof. Accordingly, the invention provide a method of preparing an extended release pharmaceutical composition including forming a core of a pharmaceutical mixture and coating the core with a coating mixture, wherein the pharmaceutical mixture includes from about 10 mg to about 500 mg of bupropion salt and a pharmaceutical acceptable polymer, and the coating mixture includes one or more surfactants and one or more aqueous water-insoluble pharmaceutical acceptable polymeric colloidal dispersions.

In one aspect, the dosage forms of the pharmaceutical composition prepared according to the invention exhibit a desired release profile for controlled release, sustained release, or extended release formulations. The term “release” is broadly defined herein as absorption or dissolution of a compound, either in vivo or in vitro. The in vivo absorption is generally performed by measuring the plasma concentration of the therapeutically active agent over a period of time. The in vitro release profile of the therapeutically active agent can be tested in a USP type 2 apparatus at about 50 rpm in about 900 ml of phosphate buffer (pH 6.8) and at 37° C. Other buffers at different pH ranges can be used herein, e.g., acetate buffer (pH 5) can be used. Any standard USP testing apparatus and conditions can be used. For example, a USP type 1 apparatus may be used at about 75 rpm under simulated intestinal buffer (SIF) or simulated gastric buffer (SGF, low pH conditions, pH at about 1.5). Various SGF buffers at various low pH ranges can be used.

In another aspect, embodiments of the invention provide a release profile of the therapeutically active agent under SIF conditions having up to about 60% release of the during the initial 2 hours, preferably from about 5% to about 40% release. The pharmaceutical composition further provides from about 10% to about 80% release of the therapeutically active agent within 4 hours, preferably from about 20% to about 75% release within 4 hours, and from about 30% to about 95% release of the therapeutically active agent within 6 hours, preferably from about 40% to about 90% release within 6 hours. Within 12 hours, the pharmaceutical composition provides no less than about 50% release of the therapeutically active agent, preferably no less than about 75% release.

In addition, embodiments of the invention provide a release profile of the therapeutically active agent under SGF conditions having up to about 20% release of the therapeutically active agent during the initial 2 hours, preferably from about 1% to about 10% release. The pharmaceutical composition further provides from about 10% to about 65% release of the therapeutically active agent within 4 hours, preferably from about 10% to about 40% release within 4 hours; more preferably from about 10% to about 25% within 4 hours; and from about 20% to about 75% release of the therapeutically active agent within 6 hours, preferably from about 30% to about 70% release within 6 hours. Within 8 hours, from about 25% to about 85% of the therapeutically active agent in the pharmaceutical composition is released, preferably, from about 40% to about 80% is released. Within 12 hours, the pharmaceutical composition provides no less than about 50% release of the therapeutically active agent, preferably no less than about 75% release.

In yet another embodiment, a method of administering a pharmaceutical composition, such as an extended release pharmaceutical composition containing bupropion salt, is provided. The method includes administering the pharmaceutical composition of the invention having a therapeutically active agent in an effective amount to treat a mammal. The pharmaceutical composition may include a core comprising the therapeutically active agent, such as bupropion hydrochloride, and a coating mixture comprising one or more surfactants and aqueous dispersions of one or more insoluble pharmaceutical acceptable polymers. For example, an extended release formulation of bupropion hydrochloride tablet prepared according to the embodiments of the invention can be used in an effective amount of about 150 mg per day for the treatment of depression or smoke cessation, such as major depressive disorders. Initial dose of about 150 mg per day may be continued for several days. In some cases, a higher dose of about 300 mg per day may be used.

### EXAMPLES

Exemplary controlled release dosage formulations are prepared and described herein. Pharmaceutical compositions having a therapeutically active agent at a concentration of from about 40% to about 80% by total weight, aqueous dispersions of a number of insoluble pharmaceutical acceptable polymers each at a concentration of from about 0.1% to about 10% of total weight, and a surfactant at a concentration of from about 0.1% to about 4.9% of total weight are formulated and tested herein. Generally, oral dosage formulations of bupropion, such as bupropion salt, bupropion hydrochloride, etc., in the form of an extended release tablet are tested in vitro for their release profile and in some cases compared in vivo to healthy human subjects with a reference formulation. The reference formulation used is the Wellbutrin® XL tablet (GlaxoSmithKline).

Bupropion hydrochloride 150 mg and 300 mg extended release tablets are prepared. Each tablet includes about 150 mg or about 300 mg of bupropion hydrochloride, Eudragit® NE 30 D (USP grade) at about 0.1% to about 10% by weight, Eudragit® L 30 D (USP grade) at about 0.01% to about 10% by weight, sodium lauryl sulfate at about 0.001% to about 4% by weight, Tween 80 (Optional) at about 0.001% to about 2% by weight, glycerol monostearate at about 0.01% to about 10% by weight, talc at about 0.001% to about 10% by weight, and optionally, an anti-sticking agent and a colorant.

Granulation: First of all, bupropion hydrochloride is charged in a vertical granulator and pre-blended for about 10 to 20 minutes. An aqueous dispersion of Eudragit® NE 30D is added to the blend and isopropylalcohol can be optionally added to achieve granulation consistency. The granules are then placed at about 50° C. until its moisture content (i.e., loss-on-drying at 105° C., 10 minutes) is below 1.5%. Once the drying is completed, granules are milled in a comill and mixed with required amounts of glycerol monostearate and talc in a V-blender.

Tabletting: The resulting granule mixture is compressed into tablet cores ( 11/32″ diameter, standard concave punch) with average hardness being between 7 and 13 Kp, each tablet core having a drug content of about 150 mg or about 300 mg. These tablet cores are then coated with a coating mixture.

First coating: The coating mixture is prepared by first dissolving sodium lauryl sulfate and Tween 80 (optional) in water and mixing the resulting surfactant-containing solution with aqueous dispersions of Eudragit® NE 30 D and Eudragit® L 30 D. The coating mixture or dispersion is then sprayed onto the tablet cores in a coating pan (O'Harra) at about 47° C.

Second coating or color coating: The coated tablet may be optionally coated again with colorants, such as Opadry, using a pan coater. Typically, a theoretical coating level of approximately 1% is obtained.

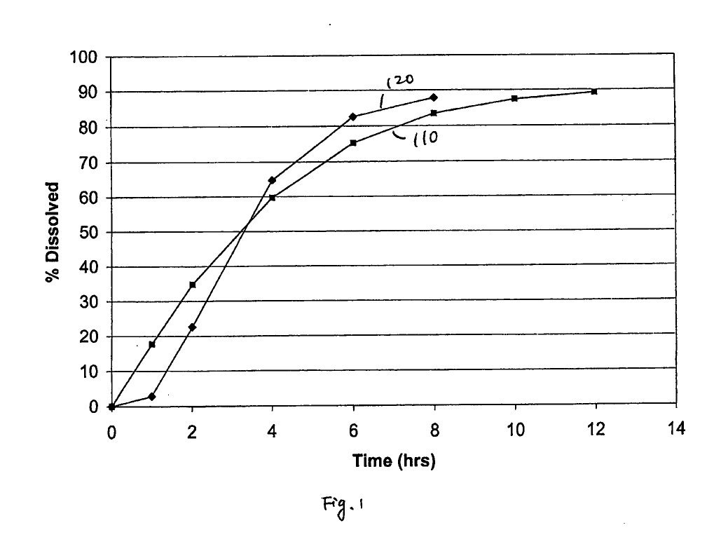

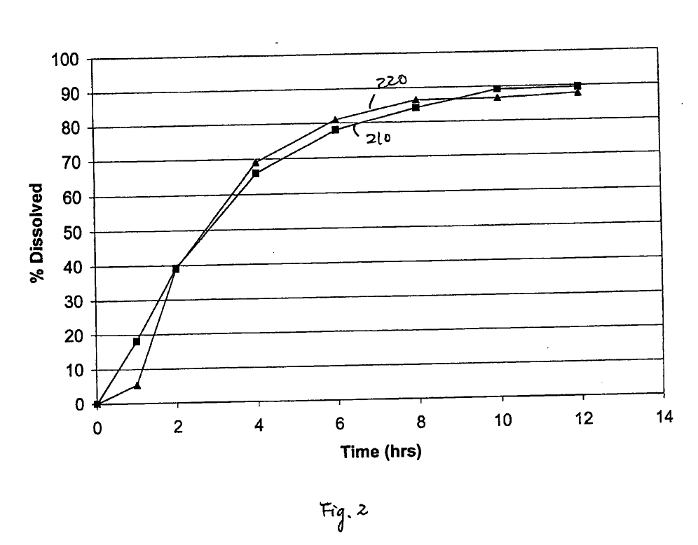

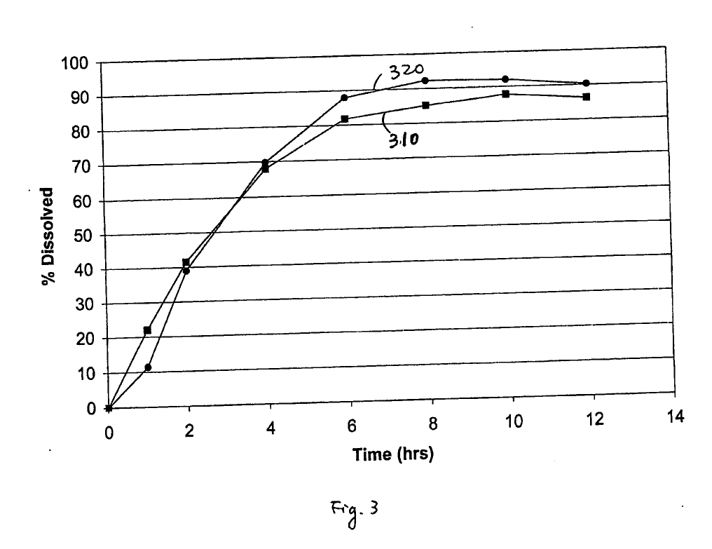

Release: The resulting tablets together with reference tablets are tested under SIF conditions and SGF conditions according to the procedure described in United States Pharmacopeia (USP). FIG. 1 is a release profile 120 of an exemplary pharmaceutical composition of about 150 mg bupropion hydrochloride tested in about 900 ml SIF buffer (pH 6.8) using an USP Apparatus 2, at a speed of about 50 rpm with paddle as compared to a release profile 110 of a reference tablet, Wellbutrin® XL tablet. FIG. 2 is a release profile 220 of an exemplary pharmaceutical composition of bupropion hydrochloride tested in about 900 ml SIF buffer (pH 6.8) using an USP Apparatus 2, at a speed of about 75 rpm with paddle as compared to a release profile 210 of a reference tablet, Wellbutrin® XL tablet. FIG. 3 is a release profile 320 of an exemplary pharmaceutical composition of bupropion hydrochloride tested in about 900 ml SIF buffer (pH 6.8) using an USP Apparatus 2, at a speed of about 100 rpm with paddle as compared to a release profile 310 of a reference tablet, Wellbutrin® XL tablet.

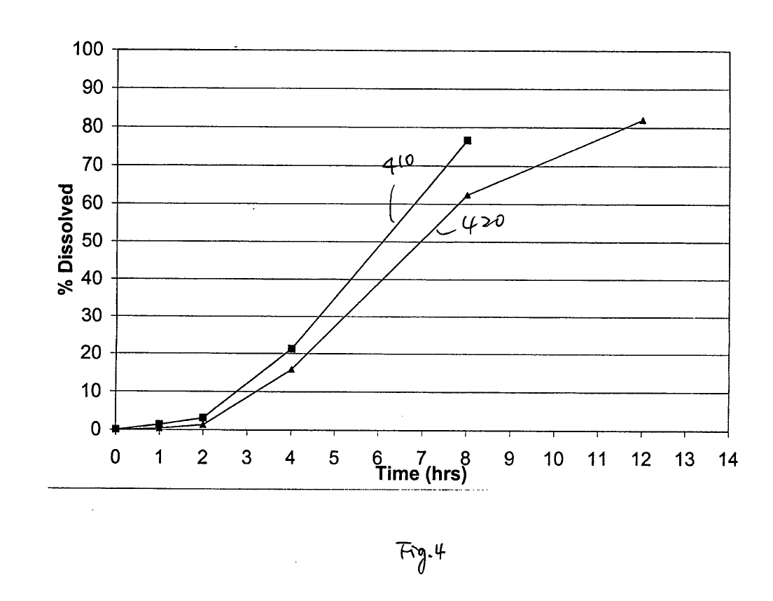

The resulting tablets together with reference tablets are also tested in about 900 ml SGF buffer (pH 1.5) according to the procedure described in United States Pharmacopeia, Apparatus 2, at various speed ranges with paddle. FIG. 4 is a release profile 420 of an exemplary pharmaceutical composition of about 150 mg bupropion hydrochloride tested in about 900 ml SGF buffer (pH 1.5) using an USP Apparatus 410 of a reference tablet, Wellbutrin® XL tablet. The results demonstrate consistent extended release profiles of bupropion hydrochloride prepared by the method and formulation of the invention, as compared to the reference tablets.

While the foregoing is directed to embodiments of the present invention, other and further embodiments of the invention may be devised without departing from the basic scope thereof, and the scope thereof is determined by the claims that follow.

What is claimed is:

1. A pharmaceutical composition, comprising:
a core comprising bupropion salt; and
a coating comprising one or more surfactants and aqueous dispersions of one or more insoluble pharmaceutical acceptable polymers.

2. The pharmaceutical composition of claim 1, wherein the bupropion salt comprises from about 10 mg to about 500 mg of bupropion hydrochloride.

3. The pharmaceutical composition of claim 1, wherein the one or more surfactants comprises from about 0.01% to about 30% by weight of the coating.

4. The pharmaceutical composition of claim 1, wherein the one or more surfactants are selected from the group consisting of ionic surfactants, anionic surfactants, non-ionic surfactants, and combinations thereof.

5. The pharmaceutical composition of claim 1, wherein the one or more surfactants are selected from the group consisting of sodium lauryl sulfate, polysorbates, Tween 20, Tween 40, Tween 60, Tween 80, and combinations thereof.

6. The pharmaceutical composition of claim 1, wherein the aqueous dispersions of the one or more insoluble pharmaceutical acceptable polymers comprise from about 5% to about 99% by weight of the coating.

7. The pharmaceutical composition of claim 6, wherein the one or more insoluble pharmaceutical acceptable polymers comprises one or more pH-independent polymers.

8. The pharmaceutical composition of claim 7, wherein the one or more pH-independent polymers comprises a swellable permeable neutral ester copolymer dispersion.

9. The pharmaceutical composition of claim 6, wherein the one or more insoluble pharmaceutical acceptable polymers comprises one or more pH-dependent polymers.

10. The pharmaceutical composition of claim 9, wherein the one or more pH-dependent polymers comprises an ionic polymer dispersion.

11. The pharmaceutical composition of claim 1, wherein the one or more insoluble pharmaceutical acceptable polymers comprises a polymer selected from the group consisting of polyacrylate, copolymer of acrylate and methacrylate, methacrylate polymer, copolymer of acrylate and methacrylate, copolymer of acrylate and methacrylate with ammonium group, copolymer of maleic anhydride and methyl vinyl ether, and combinations thereof.

12. The pharmaceutical composition of claim 1, the coating further comprising an enteric polymer selected from the group consisting of a cellulose acetate phthalate, a cellulose phthalate hydroxy propyl methyl ether, a polyvinyl acetate phthalate, a hydroxy propyl methyl cellulose acetate succinate, a cellulose acetate trimellitate, a shellac, a polyacrylate polymer, and their derivatives and combinations thereof.

13. The pharmaceutical composition of claim 1, further comprising a glidant selected form the group consisting of glycerol monostearate, talc, corn starch, metallic stearate, and combinations thereof.

14. The pharmaceutical composition of claim 1, further comprising one or more pharmaceutically acceptable excipients selected from the group consisting of fillers, extenders, binders, blending agents, emulsifiers, dispersing agents, defoamers, lubricants, nonstick agents, blenders, glidants, anti-sticking agents, colorants, diluents, dyes, pigments, dispersants, wetting agents, and their derivatives and combinations thereof.

15. A pharmaceutical composition, comprising:
a core comprising a bupropion salt and a first swellable permeable insoluble polymer dispersion; and
a coating comprising one or more surfactants and a second swellable permeable insoluble polymer dispersion.

16. The pharmaceutical composition of claim 15, wherein the first swellable permeable insoluble polymer dispersion comprises a neutral ester copolymer.

17. The pharmaceutical composition of claim 15, wherein the second swellable permeable insoluble polymer dispersion comprises a neutral ester copolymer.

18. The pharmaceutical composition of claim 15, wherein the coating further comprises an insoluble ionic polymer dispersion.

19. The pharmaceutical composition of claim 15, further comprising an insoluble enteric polymer.

20. The pharmaceutical composition of claim 15, wherein the bupropion salt comprises from about 5% to about 95% by weight, the first swellable permeable insoluble polymer dispersion comprises from about 0.01% to about 50% by weight, and the second swellable permeable insoluble polymer dispersion comprises from about 0.001% to about 50% by weight of the total weight of the pharmaceutical composition.

21. The pharmaceutical composition of claim 15, wherein the bupropion salt comprises from about 10 mg to about 500 mg of bupropion hydrochloride.

22. The pharmaceutical composition of claim 15, wherein the one or more surfactants comprises sodium lauryl sulfate.

23. The pharmaceutical composition of claim 22, wherein the one or more surfactants further comprises Tween 80.

24. A method of administering a pharmaceutical composition containing bupropion, comprising:
administering to a mammal an effective amount of the pharmaceutical composition comprising:
a core comprising bupropion salt; and
a coating mixture comprising one or more surfactants and aqueous dispersions of one or more insoluble pharmaceutical acceptable polymers.
